(19) United States
(12) Patent Application Publication
(10) Pub. No.: US 2023/0004917 A1
(43) Pub. Date: Jan. 5, 2023

(54) Performance Management System and Method

(71) Applicant: Rippleworx, Inc., Huntsville, AL (US)

(72) Inventors: Timo Sandritter, Huntsville, AL (US); Brian Hadley, Madison, AL (US); Angela Michelle Sandritter, Huntsville, AL (US); Jason P. DeVine, Alpharetta, GA (US); Amanda Bolan, Frisco, CO (US)

(73) Assignee: Rippleworx, Inc., Huntsville, AL (US)

(21) Appl. No.: 17/842,477

(22) Filed: Jun. 16, 2022

Related U.S. Application Data
(60) Provisional application No. 63/217,994, filed on Jul. 2, 2021.

(51) Int. Cl. G06Q 10/06; G01D 9/00

(52) U.S. Cl. CPC G06Q 10/06398; G01D 9/005; G06F 3/04847

(57) ABSTRACT

Systems and methods identify and trigger actions to improve performance in a role in an organization. A server computer receives input data associated with a performance of a target entity in a role in an organization from a remote device. The server computer also receives, via input to a Graphical User Interface (GUI), a set of weights for a respective set of skills for the role. The server computer computes a metric based on the input data and the weights, the metric representing the performance in the role for the target entity. The server computer identifies an action likely to improve the metric and triggers the action.

### CROSS-REFERENCES TO RELATED APPLICATIONS

This application claims the benefit of priority under 35 U.S.C. § 119 from U.S. Provisional Application No. 63/217,994, filed on Jul. 2, 2021, the disclosure of which is hereby incorporated by reference in its entirety for all purposes.

### BACKGROUND

It is increasingly popular to gather and analyze data associated with individuals. For example, devices that monitor physical states like steps taken and heart rate are widely used. Wearable devices may gather biometric information and perform simple computations based on the gathered biometric information. For example, wearable devices may compute an average number of steps taken per day, or convert a pulse rate to a heart rate. Other types of data that can be analyzed in connection with an individual include Web data, test results, and other performance data. With the proliferation of data collected about individuals, it becomes increasingly challenging to discern meaning from large amounts of data of disparate types.

### BRIEF SUMMARY

Systems and methods are described for triggering an action to improve performance in a role in an organization based on data gathered from one or more remote devices. A set of Graphical User Interfaces (GUIs) are provided to show dashboards and recommendations to improve the performance of the organization as a whole as well as individuals in the organization. GUIs can further be provided to accept user input to customize how performance is assessed, e.g., via user-configured weights, skills of interest in a particular role, and data sources used to assess the performance.

In some embodiments, a computer-implemented method comprises receiving, by a server computer from a remote device, input data associated with a performance of a target entity in a role in an organization; receiving, by the server computer via input to a Graphical User Interface (GUI), a set of weights for a respective set of skills for the role; computing, by the server computer, a metric based on the input data and the weights, the metric representing the performance in the role for the target entity; identifying, by the server computer, an action likely to improve the metric; and triggering, by the server computer, the action.

In some aspects, triggering the action comprises one or more of: modifying an entry on a calendar for the target entity to include an identified task to improve the performance; transmitting an electronic mail (email) message including the metric and at least a subset of the input data or a derivative thereof; displaying a second GUI including the metric and at least a subset of the input data or a derivative thereof; or transmitting a suggestion thereby causing the target entity to perform the action.

In some aspects, the input data includes one or more of: biometric data received from a wearable device that collected the biometric data from the target entity; performance data received from a computing device that analyzed performance of the target entity; or survey or test data received from a user device that received responses from the target entity. In some aspects, the biometric data comprises one or more of heartrate data or blood oxygenation data.

In some aspects, the method further comprises displaying the GUI, the GUI comprising one or more interactive elements for modifying the weights; receiving, via the one or more interactive elements, user input modifying the weights; and updating the weights based upon the user input.

In some aspects, computing the metric comprises identifying, by the server computer, a first skill value for a first skill for a second entity; incrementing, by the server computer, the first skill value according to a predetermined margin to generate a first baseline value; identifying, by the server computer, a second skill value for the first skill for the target entity; and computing, by the server computer, a percentage of the first baseline value for the second skill value. In some aspects, computing the metric further comprises identifying, by the server computer, a third skill value for a second skill for a third entity; incrementing, by the server computer, the third skill value according to the predetermined margin to generate a second baseline value; identifying, by the server computer, a fourth skill value for the second skill for the target entity; computing, by the server computer, a percentage of the second baseline value for the fourth skill value; and computing, by the server computer, the metric based on the percentage of the first baseline value and the percentage of the second baseline value.

In some aspects, the method further comprises displaying, via a third GUI, performance metrics for a plurality of entities including the target entity and an attribute for each entity of the plurality of entities, thereby causing a modification of the attribute for at least a subset of the plurality of entities. In some aspects, the method further comprises displaying, via a fourth GUI, an interactive element for configuring a source of the input data; and receiving, via the fourth GUI, user input configuring the source of the input data, wherein the input data is retrieved and stored by the server computer based on the configured source. In some aspects, at least a subset of the input data is retrieved from a he a Global Positioning System (GPS).

In some embodiments, a computing system comprises a processor; and a non-transitory computer readable medium operatively coupled to the processor, the non-transitory computer readable medium comprising code executable by the processor for performing any of the above methods.

### BRIEF DESCRIPTION OF THE DRAWINGS

Illustrative aspects of the present disclosure are described in detail below with reference to the following drawing figures. It is intended that that embodiments and figures disclosed herein are to be considered illustrative rather than restrictive.

FIG. 1 illustrates a schematic diagram of a system and method for analyzing and improving a performance metric for an entity according to some embodiments.

FIG. 2 illustrates a block diagram of the server computer of FIG. 1 according to some embodiments.

FIG. 3 illustrates an example of performance data.

FIG. 4 illustrates an example overview configuration process according to some embodiments.

FIG. 5A illustrates an example user interface for receiving configuration data according to some embodiments.

FIG. 5B illustrates another example user interface for receiving configuration data according to some embodiments.

FIG. 6 illustrates another example user interface for receiving configuration data according to some embodiments.

FIG. 7 illustrates another example user interface for receiving configuration data according to some embodiments.

FIG. 8 illustrates another example user interface for receiving configuration data according to some embodiments.

FIG. 9 illustrates another example user interface for receiving configuration data according to some embodiments.

FIG. 10 illustrates another example user interface for receiving configuration data according to some embodiments.

FIG. 11 illustrates an example user interface illustrating configured weights and skills, according to some embodiments.

FIG. 12 illustrates an overview process for evaluating and improving performance in a role according to some embodiments.

FIG. 13 illustrates an example user interface illustrating data ingestion according to some embodiments.

FIG. 14 illustrates another example user interface illustrating data ingestion according to some embodiments.

FIG. 15 illustrates an example user interface illustrating output information according to some embodiments.

FIG. 16A illustrates another example user interface illustrating output information according to some embodiments.

FIG. 16B illustrates another example user interface illustrating output information according to some embodiments.

FIG. 16C illustrates another example user interface illustrating output information according to some embodiments.

FIG. 17 illustrates another example user interface illustrating output information according to some embodiments.

### DETAILED DESCRIPTION

Systems and methods are described for identifying and triggering actions for improving performance in a role in an organization based on data characterizing one or more entities in the role. For example, they system may analyze performance data for players on a sports team, in roles such as linebacker and quarterback, or analyze performance data for members of a police force, in roles such as patrol officer and detective. Data associated with one or more entities (e.g., individuals or groups in the organization) is retrieved. Biometric data may be gathered from a wearable device. User feedback may also be gathered from a user interface of a user device. The system may analyze multiple types of data from multiple remote sources to compute a metric indicating a performance level in a role in the organization. This metric is computed using user-configured weights, roles, skills and/or data sources. The system triggers an action according to the computed metric, which may include transmitting advised activities, turning on or off hardware devices, and displaying a user interface with suggested actions.

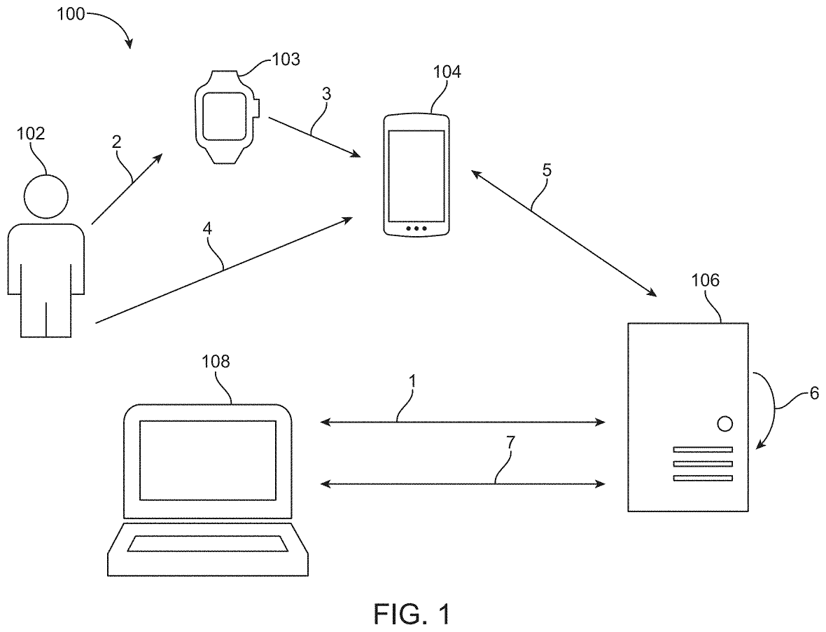

FIG. 1 illustrates a schematic diagram 100 of a computing system and method for improving performance in a role based on data characterizing one or more entities in the role according to some embodiments. The computing system may include a target entity 102, a wearable device 103, a first user device 104, a server computer 106, and a second user device 108. For simplicity of illustration, a limited number of components are shown in FIG. 1. It is understood, however, that embodiments may include more than one of each component.

The components in the computing system depicted in FIG. 1 can be in operative communication with each other through any suitable communication channel or communications network. Suitable communications networks may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. Messages between the computers, networks, and devices may be transmitted using a secure communications protocols such as, but not limited to, File Transfer Protocol (FTP); HyperText Transfer Protocol (HTTP); Secure Hypertext Transfer Protocol (HTTPS), Secure Socket Layer (SSL), and/or the like.

The server computer 106 retrieves data associated with a target entity 102 from one or more remote devices including the wearable device 103, the first user device 104, and the second user device 108. The target entity 102 can be an individual (e.g., a person, such as a member of an organization such as a sports team, company or division thereof, military organization, etc.). Alternatively, a target entity 102 may be a group of individuals, such as a division of an organization. For example, the target entity 102 can be a group of people such as the defensive players on a soccer team, the cashiers at a grocery store, etc.

The wearable device 103 may be a device wearable by a user (e.g., target entity 102) and capable of obtaining data about the target entity 102. The wearable device 103 may, for example, be a vest, watch, ring, hat, or the like. The wearable device 103 may include hardware for detecting the data about the target entity 102 such as a heart rate monitor, an oximetry sensor, a blood pressure detector, a Global Positioning System (GPS), and so forth. The data about the target entity 102 may include biometric information such as heartrate information, pulse information blood oxygen levels, and blood salinization levels. The data about the target entity 102 may include location information (e.g., as determined via GPS).

The first user device 104 may be a device operable by a user (e.g., target entity 102) and capable of executing applications. As examples, the first user device 104 may be a smartphone, a computer, a tablet, or the like. The first user device 104 may also include hardware and/or software configured to store data. The first user device 104 may also include hardware and/or software configured to receive data from the wearable device 103. The first user device 104 may include hardware and/or software configured to transmit data to the server computer 106. The first user device 104 may also be connected to the server computer 106 via a communication network. The first user device 104 may also include hardware and/or software capable of receiving user input. The first user device 104 may include a keyboard, touchscreen, microphone, and/or the like for receiving data from a user. The first user device 104 may also receive information about the target entity 102, via direct user input (e.g., the user inputs an answer to a question via a user interface displayed by the first user device 104) and/or by way of the wearable device 103 (e.g., via a wireless connection and a coupled application). In some implementations, the first user device 104 includes a GPS and/or biometric sensors as described above with respect to the wearable device 103.

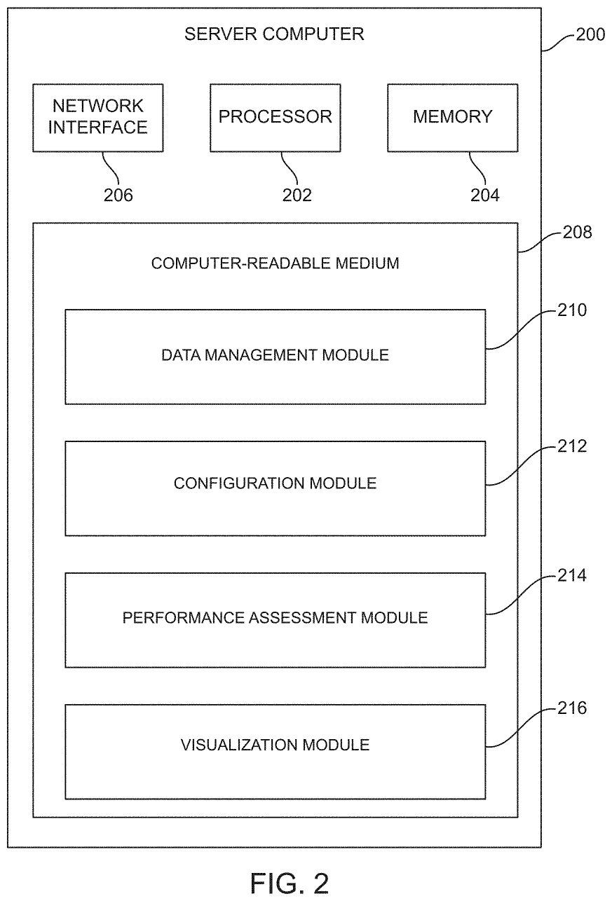

The server computer 106 may include functionality to receive and analyze data received from the first user device 104 and/or the wearable device 103. The server computer 106 may include a processor coupled to a memory, a network interface, and a computer-readable medium, as described in further detail below with respect to FIG. 2. In some embodiments, the server computer 106 is configured to gather data from the first user device 104 and/or wearable device 103, and analyze this data to evaluate performance and identify and trigger actions.

The second user device 108 may be a device operable by a user and capable of executing applications. In some embodiments, the user operating the second user device is different than the target entity 102 operating the first user device 104. For example, the second user device 108 may be operated by someone in a supervisory role with respect to the user of the first user device 104. As a specific example, target entity 102 may be an athlete, and the second user device 108 may be operated by a coach that supervises target entity 102 along with other athletes on a team. As another example, target entity 102 may be a pilot or soldier and the second user device 108 may be operated by a commander that supervises target entity 102 along with other pilots or soldiers in a division. The second user device 108 may otherwise be substantially similar to the first user device 104.

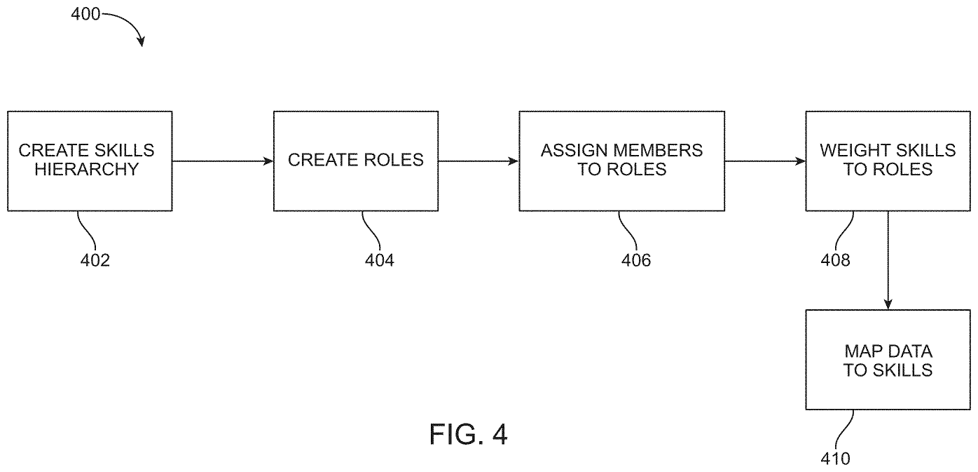

At step 1, the server computer 106 receives configuration data from the second user device 108. The configuration data can be used to establish what data to collect, and how to use and weight the data in making a performance assessment, as described herein. The configuration data can be configured by a user (e.g., an administrator, such as a coach, supervisor, doctor, etc.) via user interfaces such as those depicted in FIGS. 4-10 and 13-14.

At step 2, the wearable device 103 collects data related to target entity 102. For example, the wearable device 103 may detect a pulse of the target entity 102, which may be converted to heartrate information. As another example, the wearable device 103 may detect blood oxygenation and/or blood salinity levels of target entity 102. As another example, the wearable device 103 may detect location information associated with the target entity 102 (e.g., the GPS coordinates of the target entity 102 at one or more times). In some aspects, the wearable device 103 records a timestamp with each element of data, e.g., a set of coordinates with respective timestamps at which the coordinates were retrieved.

In some embodiments, at step 3, the wearable device 103 transmits the user data to the first user device 104. The first user device 104 may, in turn, transmit the user data to the server computer 106 at step 5. Alternatively, or additionally, the wearable device 103 may transmit the user data directly to the server computer 106. The wearable device 103 and/or the first user device 104 may analyze the data. For example, the wearable device 103 may compute a heart rate based on a detected pulse. As another example, the first user device 104 may compute a distance traveled and/or speed based on a set of GPS coordinates collected over time. In some embodiments, aggregate statistics, such as an average, minimum, maximum, event count, etc., are computed from time series data on-board the wearable device or the first user device.

At step 4, the target entity 102 may input data to the first user device 104. The target entity 102 may interact with the first user device 104 via one or more Graphical User Interfaces (GUIs). The target entity 102 may input subjective perceptions of the physical or mental state of the target entity 102. As an example, the target entity 102 takes a quiz or survey and enters various answers via a GUI, which are then stored to the first user device 104. As another example, the target entity 102 may input information about how well rested the user feels, how tired the user feels after an activity such as a workout or flying a plane, what the user has eaten that day, and so forth. In some examples, the target entity is an athlete, and the athlete inputs a numerical value representing a subjective perception of their physical exertion during an athletic session. The athlete may input, and the system may record, the subjective perception after the athletic session.

At step 5, the first user device 104 (and/or the wearable device 103) transmits information to the server computer 106. The server computer 106 may receive the information from the first user device 104 and/or the wearable device 103. The information may be time series data, i.e., a set of data with corresponding time stamps that can be used to analyze patterns in the data over time. In some embodiments, the first user device 104 transmits a first data set and second data set—e.g., two sets of time series data for different measurements. As an example, a first data set may be from the wearable device 103, e.g., heartrate, pulse, oximetry, and so forth. A second data may be from the first user device 104, e.g., information input by the user. Alternatively, or additionally, multiple data sets may be received from the wearable device 103 and/or the first user device 104. For example, heartrate and oximetry information may be received from the first user device 104 originating from the wearable device 103.

Each data set may include a plurality of data points. The data points may represent a measurement at a particular time, and may be associated with a timestamp. Each data set may also include an identifier of the user and/or user device (e.g., a universally unique identifier (UUID), user name, first and/or last name, nickname, Internet Protocol (IP) address, and so forth).

Alternatively, or additionally, the server computer 106 may receive performance data from a computing device that analyzed performance of the target entity. As specific examples, the performance data can be player statistics for a sports player (e.g., passing accuracy, shooting percentage, and so forth) or efficiency statistics for an office worker (e.g., time spent typing in documents or sending emails in a given day). In some implementations, the computing device applies one or more machine learning models to identify the performance data. For example, the computing device applies a machine learning model trained to identify and count each time a tennis player hits a ball in practice footage. The number of times hitting the ball is then compiled and sent to the server computer 106. The machine learning model may, for example, be a neural network.

As another example, the server computer 106 may receive user information from a computer operated by a doctor administrating tests to the target entity 102. As another example, the server computer 106 may receive information from a vehicle operated by the target entity 102. The information may relate to a status of the vehicle. For example, the target entity 102 may operate an airplane, and the airplane may transmit altitude information, speed information, GPS information, and so forth. As other examples, a vehicle (e.g., a car, truck, tank, or submarine operated by the user) may transmit vehicle information to the server computer 106.

At step 6, the server computer 106 analyzes the data received from the wearable device 103 and/or first user device 104 according to the configuration data received from the second user device 108. The server computer 106 may perform statistical operations on the received data such as sum, count, average, and standard deviation. The server computer 106 may correlate received data. For example, the server computer 106 correlates a first data set received from the wearable device 103 and a second data set received from the first user device 104 based on timestamps, user identifiers, and/or device identifiers. As a specific example, the server computer may correlate a heart rate and an oximetry level based on same or similar timestamps (e.g., within one second or ten seconds of one another). The data points in the first data set and the second data set may be correlated over time to analyze how the first data set and the second data set relate to one another (e.g., time series data).

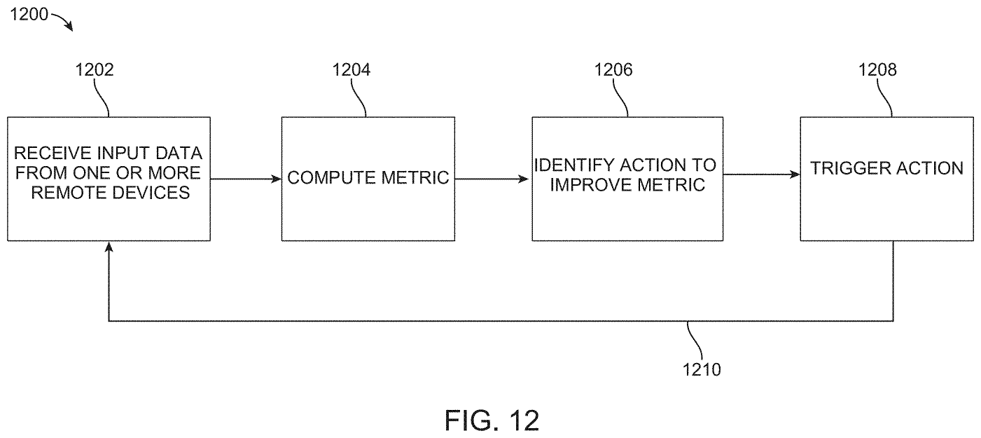

In some embodiments, at step 6, the server computer 106 computes a metric based on the received data and the configuration data. The metric may represent how the target entity 102 is performing in a role. In some implementations, the metric represents how the target entity 102 is performing in comparison to other entities in that role. Techniques for computing the metric are described in further detail with respect to FIG. 12.

At step 7, the server computer 106 identifies and triggers an action based on the metric. For example, the server computer 106 transmits a message to the second user device 108 and/or the first user device 104. The message may be in the form of a push notification, an email, a text message, and/or the like. For example, the server computer 106 transmits, to the first user device 104 and/or second user device, an electronic mail (email) message including the metric and at least a subset of the input data or a derivative thereof. As a specific example, the target entity is a baseball player, and the metric is 5 out of 10, indicating that the target entity's performance has room for improvement. The email includes the metric, as well as a derivative of a subset of the input data in the form of an average sprinting speed of the target entity, which is significantly lower than average.

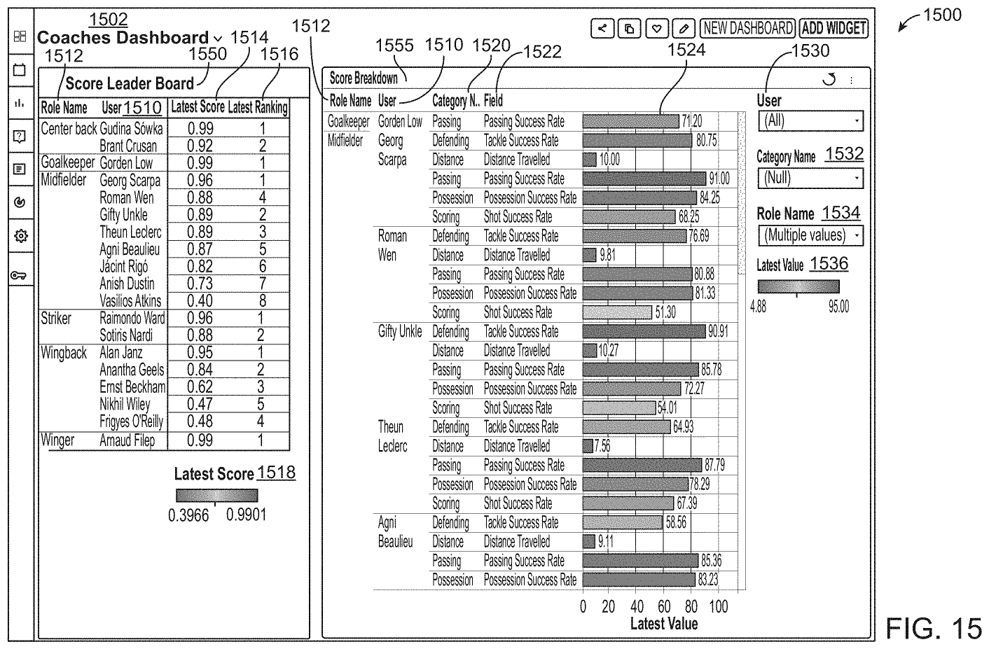

Alternatively, or additionally, the server computer updates a user interface displayed on the second user device 108 and/or the first user device 104. The server computer 106 may cause display of information via a user interface. The server computer 106 may transmit instructions to the second user device 108 and/or the first user device 104, thereby causing the second user device 108 and/or the first user device 104 to display a Graphical User Interface (GUI) including the metric and at least a subset of the input data or a derivative thereof via the first user device and/or second user device. As illustrated in FIGS. 15-17, the server computer may cause display of information indicating the metric, information involved in computing the metric, and/or suggested actions determined based upon the metric.

In some implementations, the server computer 106 modifies an entry on a calendar for the target entity to include an identified task to improve the performance. For example, the target entity is a race car driver and the metric is low due to relatively poor historical performance when passing another car on the race track. The driver's schedule, in the form of a digital calendar, is updated to include additional passing practice. As another example, analysis of an athlete's performance and the athlete's subjective perceptions of how tired he feels results in a low metric due to the athlete being on the verge of an injury. The athlete's calendar is updated to include more rest and stretching. As another example, the target entity is a police officer and the metric is based on correlating schedules to mental wellbeing. The server computer 106 determines that the target entity is on third shift too long, leading to mental and physical problems. The calendar is updated to move the officer from the third shift to mitigate these effects.

In some implementations, the server computer 106 transmits a suggestion, thereby causing the target entity to perform an action to improve the performance. For example, one or more of the above alerts can be transmitted, along with a suggestion such as going to a class, talking to a therapist, increasing a certain type of training, etc. The target entity follows the suggestion, and then further data is collected, the metric is recomputed, and the metric has increased, indicating an improvement in performance.

In some implementations, the server computer 106 causes a modification to equipment. This may include activating or deactivating the equipment. For example, the target entity is a law enforcement officer and the metric indicates that the target entity is underperforming. The corresponding action is turning on a body camera worn by the target entity. The server computer may transmit a signal over a wireless network causing the equipment to be activated or deactivated. Alternatively, or additionally, the equipment is modified by changing the issued equipment—e.g., a law enforcement officer is issued a body camera, a sports player is issued new shoes, and so forth.

FIG. 2 illustrates a server computer 200 according to some aspects of the disclosure. The server computer 200 may, e.g., be the server computer 106 of FIG. 1. The server computer 200 includes functionality to receive and analyze data received from the first user device 104, second user device 108, and/or the wearable device 103. The server computer 200 includes a processor 202 coupled to a memory 204, a network interface 206, and a computer-readable medium 208.

The memory 204 can be used to store data and code. The memory 204 may be coupled to the processor 202 internally or externally (e.g., cloud based data storage), and may comprise any combination of volatile and/or non-volatile memory, such as RAM, DRAM, ROM, flash, or any other suitable memory device. The memory 204 may store user data collected in association with one or more users over time.

The processor 202 may comprise one or more processors, application specific integrated circuits (ASICs), or field programmable gate arrays (FPGAs). The processors may include be single core or multicore processors. In some embodiments, processor 202 can include one or more special purpose co-processors such as graphics processors, digital signal processors (DSPs), or the like. In some embodiments, the processor 202 can be implemented using customized circuits, such as application specific integrated circuits (ASICs), or field programmable gate arrays (FPGAs).

In some embodiments, the processor 202 can execute instructions stored in memory 204 or on computer-readable medium 208. In various embodiments, the processor 202 can execute a variety of programs or code instructions and can maintain multiple concurrently executing programs or processes. At any given time, some or all of the program code to be executed can be resident in memory 204 and/or on computer-readable medium 208 including potentially on one or more storage devices. Through suitable programming, processor 202 can provide various functionalities described herein.

The network interface 206 may include an interface that can allow the server computer 200 to communicate with external computers. The computer-readable medium 208 is a non-transitory computer-readable medium and may include software code stored as a series of instructions or commands. The computer-readable medium 208 may comprise code, executable by the processor, to implement methods as described herein.

In some aspects, the computer-readable medium includes a data management module 210, a configuration module 212, a performance assessment module 214, and a visualization module 216.

The data management module 210 includes code for importing, storing, and organizing data. In some embodiments, the data management module 210 is configured to retrieve data from one or more external devices (e.g., wearable devices, user computing devices, other server computers, etc.). The data management module 210 may further be configured to store the data in an organized fashion (e.g., in chronological order and/or in association with a user identifier or device identifier).

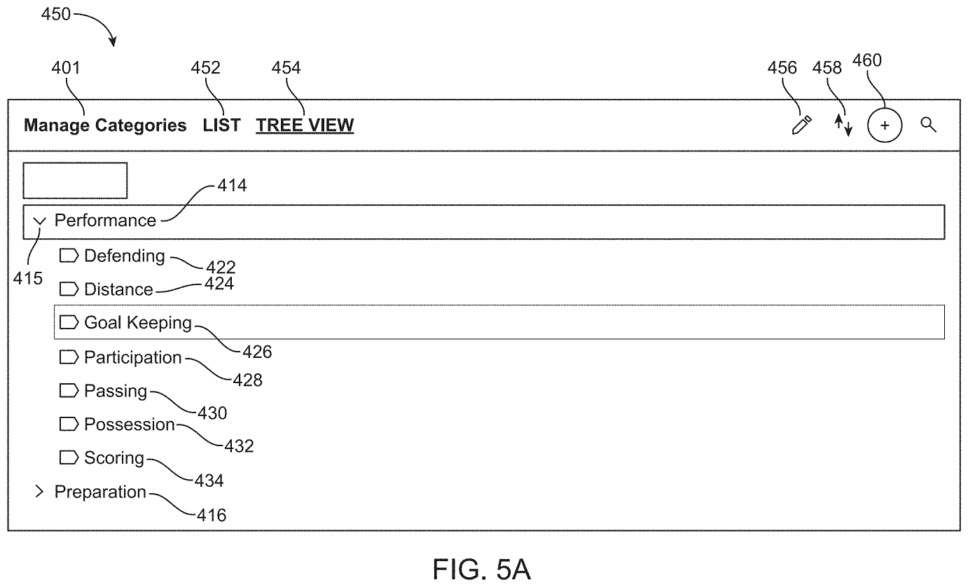

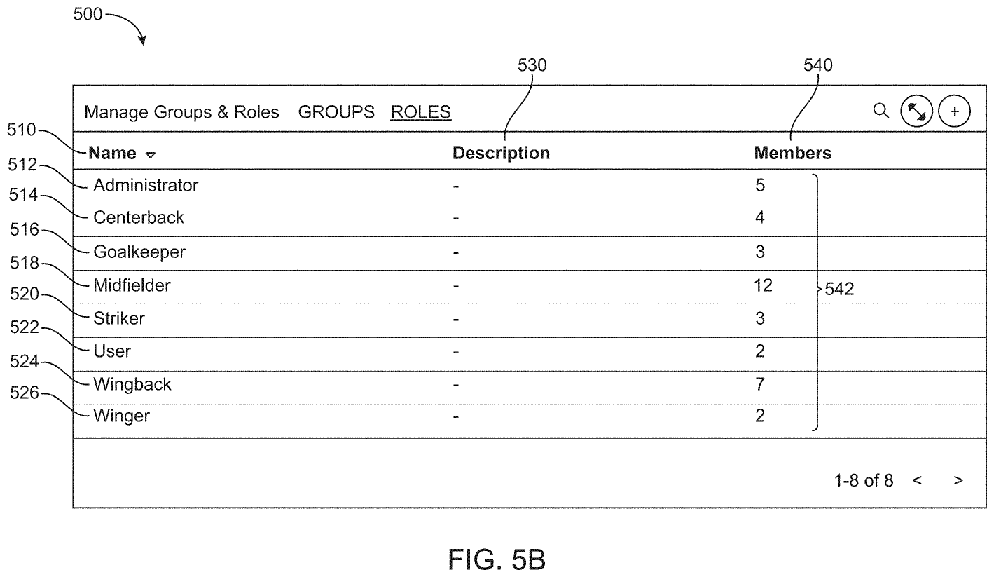

The configuration module 212 includes functionality to identify and manage attributes configured by a user. The configuration module 212 may identify attributes to be configured by a user such as weights, categories, roles, and data sources, as described herein. The configuration module 212 may prepare interface elements for display to guide a user to provide configuration values (e.g., as shown in FIGS. 5A-10). The configuration module 212 may further apply the configuration values to customize the data structures and analytics based on user input.

The performance assessment module 214 includes code configured to compute a metric indicative of a target entity's performance, and identify and trigger actions to perform the performance. The performance assessment module 214 may include code configured to retrieve data for an entity, apply user-configured attributes such as weights, and compute a performance metric indicative of the target entity's performance in a role based on the data and the user-configured attributes. The performance assessment module 214 may further include code configured to identify actions for improving the performance metric and the performance of the target entity in a role. For example, the performance assessment module may 214 include functionality for traversing a database that maps different skills or aspects of a role to different activities to be performed to identify actions that will improve different aspects of a role. As specific examples, an entity can take a typing class to improve typing or do batting practice to improve hitting.

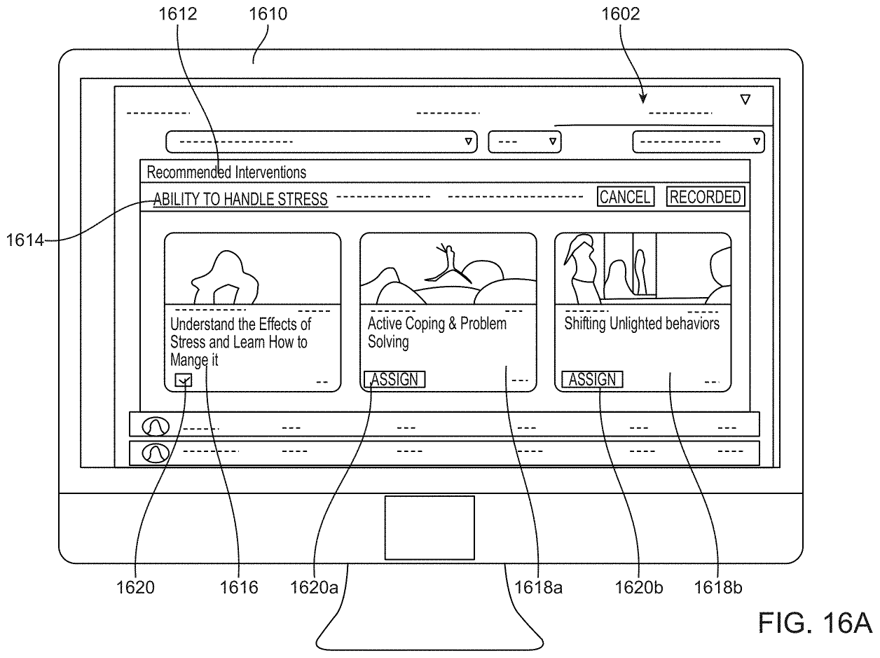

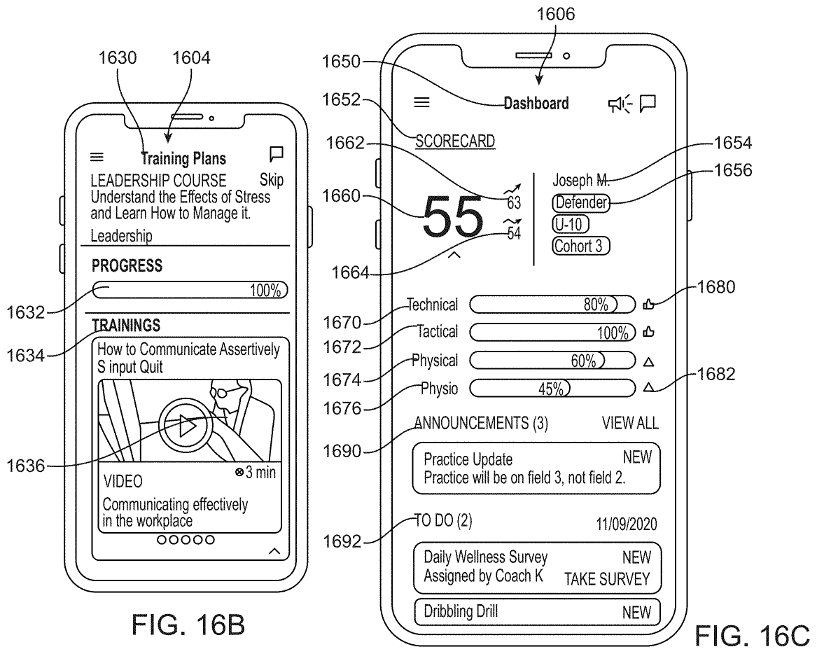

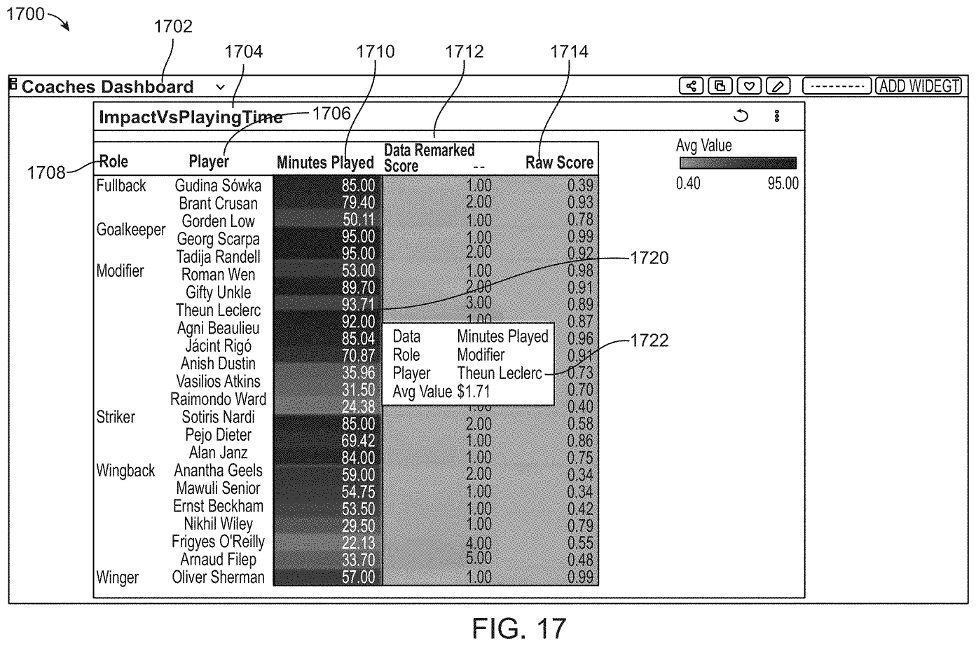

The visualization module 216 includes functionality to generate visualizations, which may include user interfaces illustrating the performance of entities in an organization. This can include coaches dashboards showing performance metrics for team members as shown in FIGS. 15 and 17 and interfaces for guiding a user to engage in activities to improve performance, as shown in FIGS. 16A, 16B, and 16C. The visualization module 216 may further include code configured to generate and cause display of interface views for configuring how and for what entities the performance metrics are computed, e.g., as illustrated in FIGS. 5A-11 and 13-14.

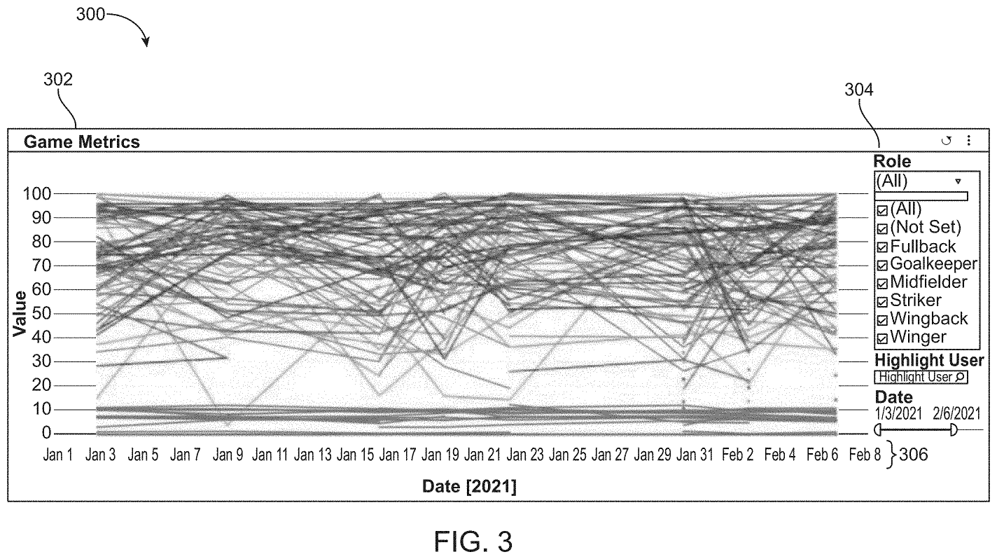

FIG. 3 illustrates an example of performance data 300. As noted above, performance data can come from a variety of sources and entities, which makes it difficult to discern meeting from these large amounts large amounts of data of disparate types. FIG. 3 shows game metrics 302, which include different data points gathered based on players' performance in a sports game. The data covers different roles 304 and dates 306. This raw data is not instructive as to how each player is doing or how to improve the players' performance. The techniques described below can be used to discern meaning from such user performance data 300 and use it to trigger actions to improve the performance of entities in roles.

FIG. 4 illustrates an example configuration process 400 according to some embodiments. As noted above with respect to FIG. 1, in some embodiments, the system presents one or more user interfaces that prompt a user to provide input configuring to what extent different factors will affect the performance metric and/or ensuing action. The process 400 may include a user (e.g., an administrator such as a coach, employer, etc.) establishing configuration data including skills, roles, members, and weights. The configuration data may be received by a server computer 106 from a user device (e.g., second user device 108) over a communication network.

At step 402 a skills hierarchy is created. As shown in FIG. 5A, the system may present a user interface including different skills and skill categories. A skill is a particular activity to be monitored and/or improved, such as defending, distance, goal keeping, etc. A skill category is a category of skills, such as performance, preparation, etc. A skills hierarchy establishes categories, and potentially subcategories, and what skills fall in what category or subcategory. User input can be received to configure particular skills and corresponding skill categories. In some aspects, based on the received user input, the system stores a data structure nesting the skills within the skill categories.

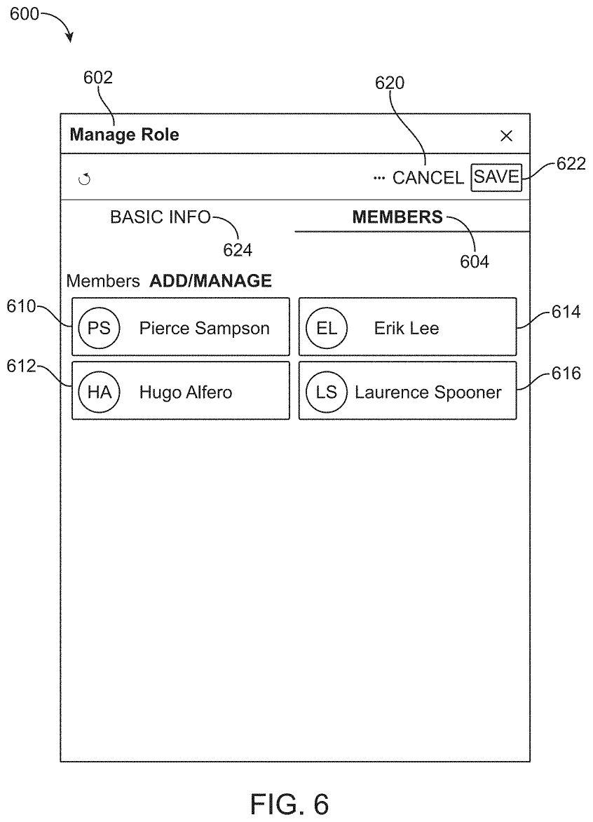

At step 404, roles are created. As shown in FIG. 6, the system may present a user interface including functionality to enter (e.g., by typing, selecting from a drop-down or other interface element, etc.) different skills and subskills. User input can be received to configure particular skills and corresponding subskills.

At step 406, members are assigned to roles. As shown in FIG. 5B, the system may present a user interface including functionality to add or manage members for a given role. A role is a position in an organization. For example, in a soccer team, roles include goalkeeper, striker, and the like; in a business, roles may include secretary, accountant, and so forth. The user interface may include one or more text entry fields for accepting typed user input, a drop-down for facilitating user selection of one of several options, functionality to drag and drop a member to a different role, or other suitable interface elements. User input can be received to assign members (e.g., different entities, such as players on a team, employees at a workplace, etc.) to a particular role.

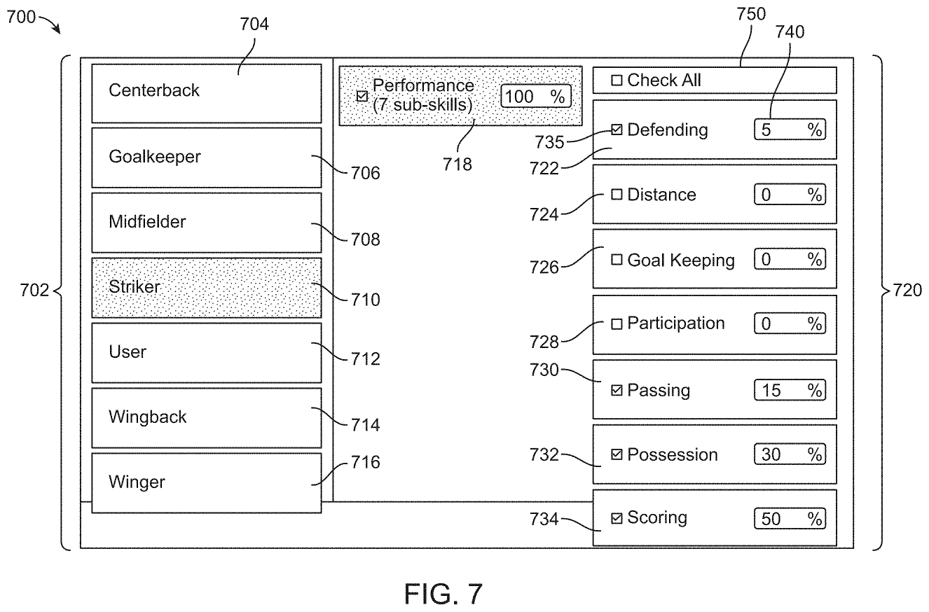

At step 408, skills are weighted to roles. For a given role, different skills may apply. For example, for an athlete, passing and kicking may be applicable skills. The system may prompt a user to configure weights that establish how much of an impact each skill has on an overall performance metric. As shown in FIGS. 7-9, the system may present user interface views including functionality to adjust these weights (e.g., by typing, selecting from a drop-down or other interface element, etc.) for skills and/or skill categories corresponding to a given role. User input can be received to adjust a weight for each skill, which will affect how much impact each of the skills has on determining a recommended course of action for entities for a particular skill category in a particular role.

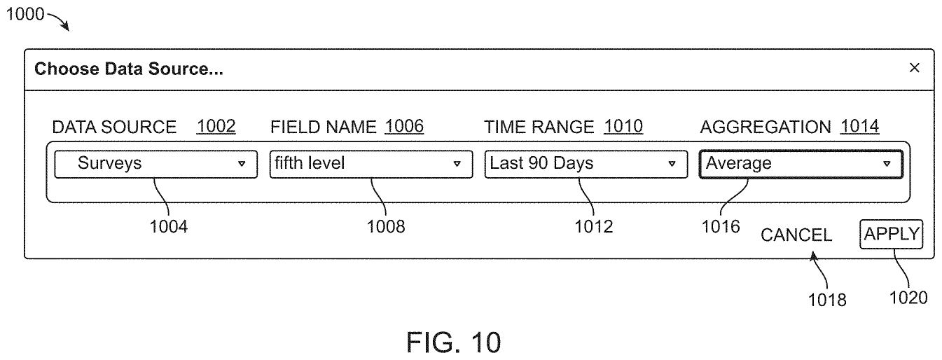

At step 410, data is mapped to skills. Via a user interface, the system prompts a user to establish data sources for the respective skills. For example, depending on the skill and the resources available, appropriate data sources may include wearable biometric sensors, user input to a user device, video or image data, GPS data, and so forth. As shown in FIG. 10, the system can present a user interface that accepts user input configuring data sources and data fields for ingesting and storing input data for use in determining the scores and metrics described herein. The process 400 of FIG. 4 is used to accept and digest user input to configure different skills for roles, and weight how the skills are used to manage performance in the respective roles.

FIG. 5A illustrates an example user interface 450 for receiving configuration data according to some embodiments. The user interface 450 can be used to configure a skills hierarchy (e.g., as in step 402 of the process 400 of FIG. 4). The user interface 450 enables managing skill categories, as indicated by the manage categories heading 401. The user interface 450 includes selectable icons list 452, for displaying the categories and skills in a list view, and tree view 454, for displaying the categories and skills in a tree view 454. In this example, the tree view 454 has been selected.

The user interface 450 depicted in FIG. 5A displays the skill categories performance 414 and preparation 416. For a given category, a drop-down icon such as drop-down icon 415 is displayed. As shown in FIG. 5A, the drop-down icon 415 for performance 414 has been selected, revealing the current skills in the performance 414 category. The skills in the performance 414 category are defending 422, distance 424, goalkeeping 426, participation 428, passing 430, possession 432, and scoring 434. These skills affect the success of one or more entities in the performance 414 skill category. The skill categories of performance 414 and preparation 416 both affect the success of one or more entities in a corresponding role (e.g., players on a soccer team).

In some implementations, a user can manipulate the skills and skill categories displayed (e.g., by dragging and dropping, typing, etc.) to configure skills and skill categories for a given role. The user interface 450 includes an editing icon 456. When a user interacts with the editing icon 456, the user interface 450 can transition to a view for user modification of the skills and/or categories. A sorting icon 458 is provided for sorting the skills and/or categories (e.g., alphabetically, by recency of addition to the list, in order of their weights, etc.). The user interface 450 further includes an add button 460, which, when selected via user interaction, transitions to a view for accepting user input to add skills and/or categories. In some implementations, the categories and skills are initially displayed based on defaults, which an be adjusted by the user. Alternatively, the categories and/or skills can be provided by a user from scratch.

FIG. 5B illustrates another example user interface 500 for receiving configuration data according to some embodiments. The user interface 500 can be used to configure roles (e.g., as in step 404 of the process 400 of FIG. 4). The user interface 500 displays different roles in an organization. In this example, the roles are listed under a drop-down menu labeled name 510. The roles in this example are administrator 512, centerback 514, goalkeeper 516, midfielder 518, striker 520, user 522, wingback 524, and winger 526, corresponding to different roles on a soccer team. Other examples or roles include positions within a company, such as secretary, banker, human resources representative, etc., or positions within a military organization, such as pilot, soldier, general, etc. The user interface 500 includes a column for description 530, which can include information describing the different roles (blank and yet-to-be configured in this example). The user interface 500 includes a column for members 540, which shows how many members are assigned to each role via respective numbers of members 542. In some implementations, a user (e.g., an administrator) can configure what members are assigned to what roles, as well as add or edit descriptions. In some implementations, the numbers listed under members are linked to the user interface 600 of FIG. 6, which can be used to view and/or edit the members in a given role. The role may control what skills contribute to a metric for a given member. For example, athletic skills are key for centerback 514 and goalkeeper 516, but less important for administrator 512.

FIG. 6 illustrates another example user interface 600 for receiving configuration data according to some embodiments. The user interface 600 can be used to assign members 604 to a role 602 (e.g., as in step 406 of the process 400 of FIG. 4). The user interface 600 is role specific. For example, using an interface such as that depicted in FIG. 5B, a particular role can be selected, and the user can drill down into what users are placed in that role 602. In some implementations, the user interface 600 is a modal that is overlaid over another interface such as the user interface 500 of FIG. 5B.

In this example, the user interface 600 displays a list of members 604 currently assigned to the role 602 of centerback—Pierce Sampson 610, Erik Lee 614, Hugo Alfero 612, and Laurence Spooner 616. User input can be received which the system uses to add or remove entities from the member list for a given role. The user interface 600 further includes a save button 622 for saving changes, a cancel button 620 for canceling changes, members 604 tab (selected in this example) for viewing or configuring members, and a basic info tab 624 for displaying additional information about a role.

FIG. 7 illustrates another example user interface 700 for receiving configuration data according to some embodiments. The user interface 700 can be used to weight skills and select skills for roles (e.g., as in step 408 of the process 400 of FIG. 4). The user interface 700 displays a list of roles 702 on the left hand side—centerback 704, goalkeeper 706, midfielder 708, striker 710, user 712, wingback 714, and winger 716. A user can interact with the user interface 700 to select a role to configure or view. As shown in FIG. 7, striker 710 has been selected and is shaded. The user interface 700 shows a list of available skills 720, with a selectable check box for each available skill. For the selected role of striker 710, the skill category of performance 718 is available and selected (as indicated by the checkmark). Next to the skill category of performance 718 is a configurable percentage box 719. In this example, since performance 718 is the only skill category assigned to the striker 710 role, it accounts for 100% (e.g., for contributing to the metric determination as described herein).

For the selected skill category of performance 718, the right hand side lists the available skills 720—defending 722, distance 724, goal keeping 726, participation 728, passing 730, possession 732, and scoring 734. A user can check one or more of the skills 720 for weighting (e.g., for contributing to the metric determination as described herein). As shown in FIG. 7, the skills of defending 722, passing 730, possession 732, and scoring 734 have been selected using corresponding checkboxes. When a checkbox 735 is activated, a text field 740 for the corresponding skill 720 is activated, so that a user can enter or edit a percentage in the text field 740. The remaining skills of distance 724, goal keeping 726, and participation 728 have not been selected and their weighting percentages are greyed-out and fixed to 0. The selected skills 720 are assigned default or user-configured weights—defending has been set to 5%, passing has been set to 15%, possession has been set to 30%, and scoring has been set to 50%. A user can interact with the text fields 740 (e.g., by typing or using a drop-down menu) to change the weights. In some implementations, when one weight is changed, the other weights are automatically changed (e.g., by an equal amount) such that the weights of the selected skills add to 100% or another configured percentage for the overall skill. The user interface 700 also includes a box labeled “check all” 750, which can be selected to include all available skills.

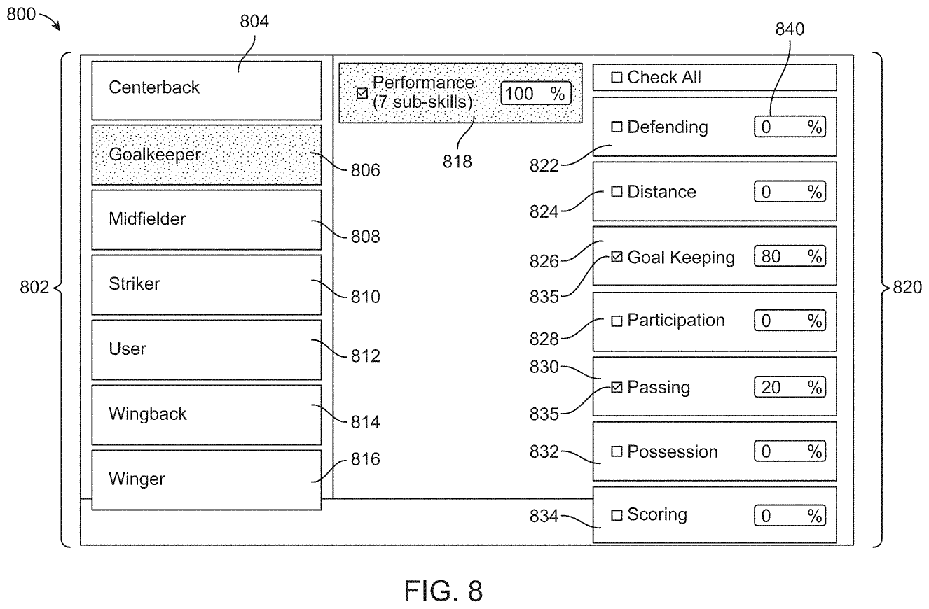

FIG. 8 illustrates another example user interface view 800 for receiving configuration data according to some embodiments. The user interface view 800 may correspond to another view of the user interface 700 of FIG. 7, when a different role 802 has been selected. Similarly to the user interface 700 of FIG. 7, the user interface view 800 can be used to weight skills to roles (e.g., as in step 408 of the process 400 of FIG. 4). In this case, the role of goalkeeper 806 has been selected from the roles 802 of centerback 804, goalkeeper 806, midfielder 810, user 812, wingback 814, and winger 816. As in FIG. 7, the listed skills 820 include defending 822, distance 824, goal keeping 826, participation 828, passing 830, possession 832, and scoring 834. Based on the selected role of goalkeeper 836, different skills have been selected that are more appropriate for a goalkeeper 836, as indicated by the activated checkboxes 835. In this case, goal keeping 826 and passing 830 are selected to contribute, and defending 822, distance 824, participation 828, possession 832, and scoring 834 are not selected to contribute. Similarly to the user interface 700 of FIG. 7, in the user interface view 800 of FIG. 8, each selected skill 820 includes a weight or percentage adding up to 100% total for the selected performance skill category, and the respective weights can be adjusted via text fields 840.

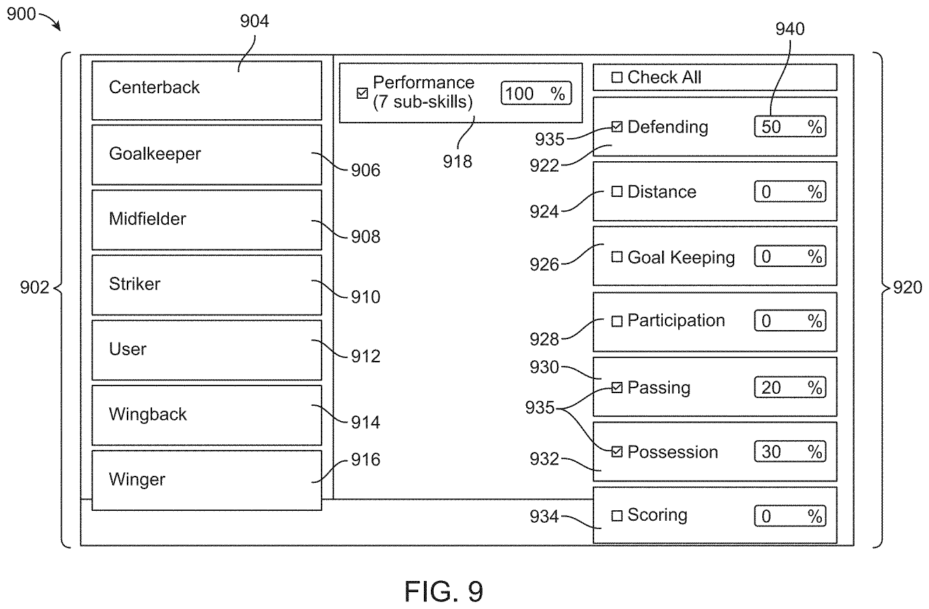

FIG. 9 illustrates another example user interface view 900 for receiving configuration data according to some embodiments. The user interface view 900 may correspond to another view of the user interface 700 of FIG. 7, when a different role 902 has been selected. Similarly to the user interface 700 of FIG. 7, the user interface view 900 can be used to weight skills to roles (e.g., as in step 408 of the process 400 of FIG. 4). In this case, the role of centerback 904 has been selected, and the skills 920 selected (indicated by activated checkboxes 935) are different. With centerback 904 selected, defending 922, passing 930, and possession 932 are selected to contribute to further analysis for users in the centerback 904 role 902, and distance 924, goal keeping 926, participation 928, and scoring 934 are not selected to contribute. Similarly to the user interface 700 of FIG. 7, in the user interface view 900 of FIG. 9, each selected skill 920 includes a weight or percentage adding up to 100% total for the selected performance skill category, and the respective weights can be adjusted via text fields 940.

FIG. 10 illustrates another example user interface 1000 for receiving configuration data according to some embodiments. The user interface 1000 can be used to configure a data source. For example, for a given type of data, different data sources 1002 can be chosen, such as surveys 1004, a particular wearable device, and so forth. The user interface 1000 is labeled Choose Data Source 1001 and includes drop-down menus labeled Data Source 1002, Field Name 1006, Time Range 1010, and Aggregation 1014. The Data Source 1002 drop-down menu can be used to select a data source (e.g., from a biometric device, survey, test, performance monitoring computing device, etc.). In this example, Surveys 1004 is the selected data source. Based on this configuration data (e.g., for a particular data field or skill), data will be retrieved from the selected data source. The Field Name 1006 drop-down menu can be used to select a name for the configured data field (e.g., fifth level 1008, as shown). The Time Range 1010 drop-down menu can be used to select a time range (e.g., last 90 days 1012 as shown). Based on the configured time range, data will be retrieved for that time range. The Aggregation 1014 drop-down menu can be used to select an aggregation method for aggregating the data (e.g., averaging 1016, as is shown). Based on the configured aggregation method, the retrieved data will be averaged (or added, the mean or median computed, etc.). The user interface 1000 further includes a cancel button 1018 for canceling any changes entered and an apply buttons 1020 for applying any changes entered.

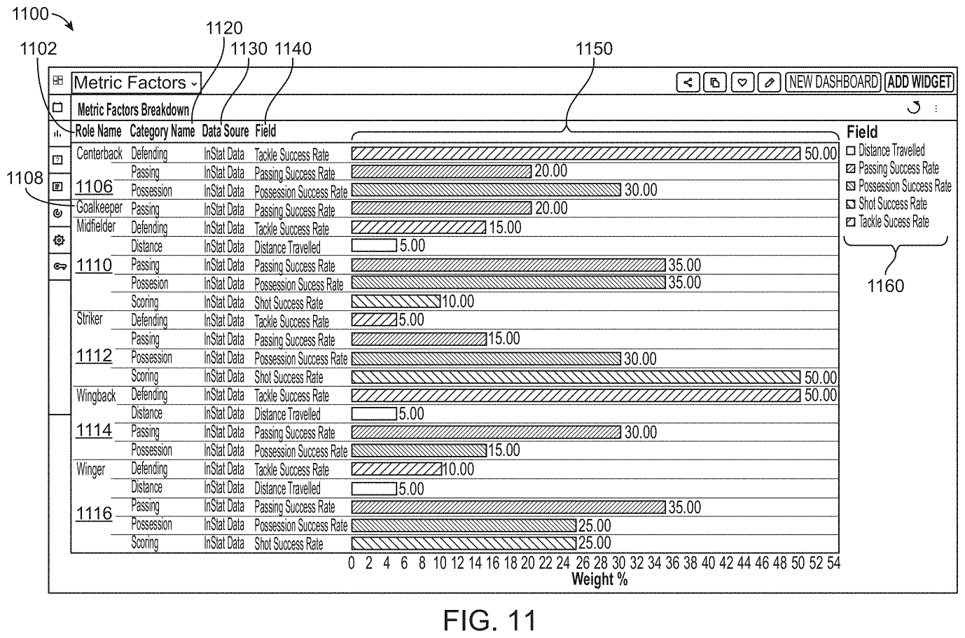

FIG. 11 illustrates an example user interface 1100 illustrating configured weights and skills, according to some embodiments. This represents what skills will be used, and with what weights, for each role in an organization, in computing a metric as described herein. This user interface 1100 can be used to show the results of the user configurations applied via the process 400 and the user interfaces 450-1000 described above. The user interface 1100 displays a list of roles, labeled Role Name 1102 and including the roles of Centerback 1106, Goalkeeper 1108, Midfielder 1110, Striker 1112, Wingback 1114, and Winger 1116. For each role, different categories 1120 and data sources 1130 are shown. The user interface 1000 further displays a list of fields 1140, which correspond to the skills selected for each respective role. For each field or skill is a bar graph 1150 showing the weight assigned for that skill for the given role—e.g., 50% tackle success rate, 20% passing success rate, and 30% possession success rate for centerback, and so forth. Each field or role is also categorized, as shown with different, as explained by the Field legend 1160.

FIG. 12 illustrates an overview process for evaluating and improving performance in a role according to some embodiments. The process 1200 may include ingesting data, analyzing that data, and using that data to identify and trigger actions to improve performance in a role. The process 1200 can be performed by the server computer 106 in cooperation with other devices in the computing system of FIG. 1.

At step 1202, the server computer receives input data from a remote device. The server computer can retrieve the data (e.g., over a communication network) from one or more remote devices, such as wearable devices, mobile devices, and/or computing devices. As described above with respect to FIG. 1, data can be ingested by the system from one or more remote devices. For example, the server computer retrieves input data including one or more of: biometric data received from a monitoring device that collected the biometric data from a target entity, performance data received from a computing device that analyzed performance of the target entity, and/or survey or test data received from an entity device that received responses from the target entity.

The input data is associated with a performance of the target entity in a role in an organization. For example, for an athlete, running speed and other athletic criteria may be relevant to their performance in their position on the sports team. For a role in a business, different skills may apply, such as typing speed and interpersonal and other skills. The relevant data, based on the role of the target entity in the organization, is retrieved from one or more remote computing devices.

Various types of data can be gathered from various types of remote computing devices. As an example, the server computer retrieves, from a wearable device with one or more biometric sensors, biometric data such as heartrate data, blood oxygenation data, or the like (i.e., heartrate or pulse when performing the action of interest). As another example, the server computer retrieves at least a subset of the input data from a Global Positioning System (GPS). The server computer may retrieve GPS data from a user device or wearable device associated with the user. The server computer may then analyze position and time data to identify an average speed of the user over a time interval. As another example, the server computer retrieves, from a computing device associated with the target entity, answers to survey questions (e.g., “How tired did you feel after running sprints today?”, “Do you find it difficult working in groups?”, etc.). As another example, the data is gathered from an external computing device that performs machine learning-based analysis of video footage of each entity on the field.

As described above, the different skills assessed, based on the role of the target entity, can be configured by a user and/or set to default values based on the role and organization at issue. For example, as described above with respect to FIGS. 4-11, user input can be accepted via a GUI to configure what skills are assessed for what role (e.g., for the role of forward, passing and kicking can be set to user-configured and/or default skills for that role).

In some embodiments, the server computer receives user input via a GUI (e.g., a first GUI) to establish a set of weights for the respective set of skills for the role. The computing system (e.g., a user device) displays the GUI, which includes one or more interactive elements (e.g., text entry fields, sliders, drop-down menus, etc.) for modifying the weights. The computing system receives user input modifying the weights via the one or more interactive elements, and updates the weights based on the user input. As an example, for the role of striker, defending, passing, possession, and scoring are skills of interest. As shown in FIGS. 7-9, a user can interact with the GUI to establish weights for each of these skills so that each weight contributes to a certain percentage of an overall performance metric. This allows the user to adjust the computations to tailor performance assessment and improvement to what is important in that role and that organization.

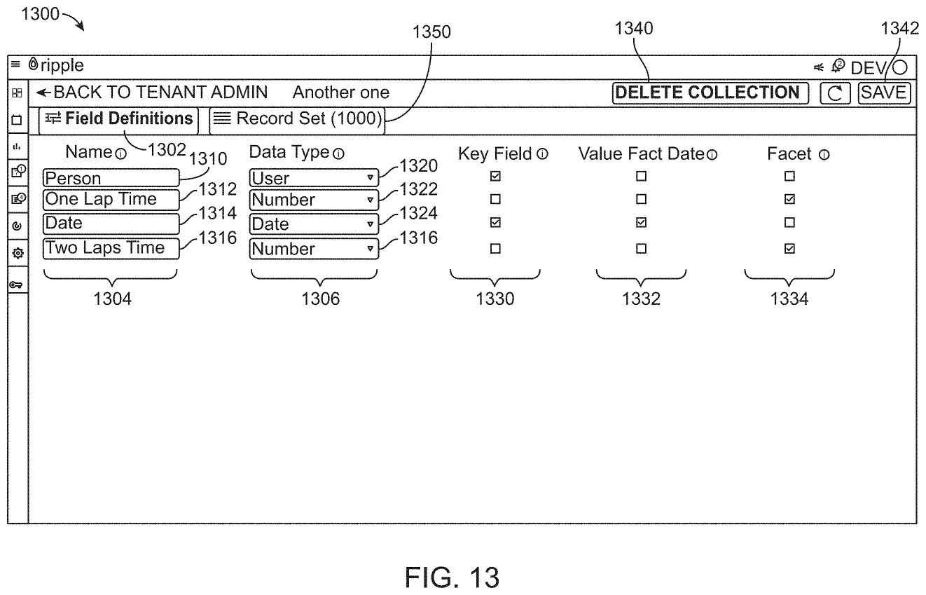

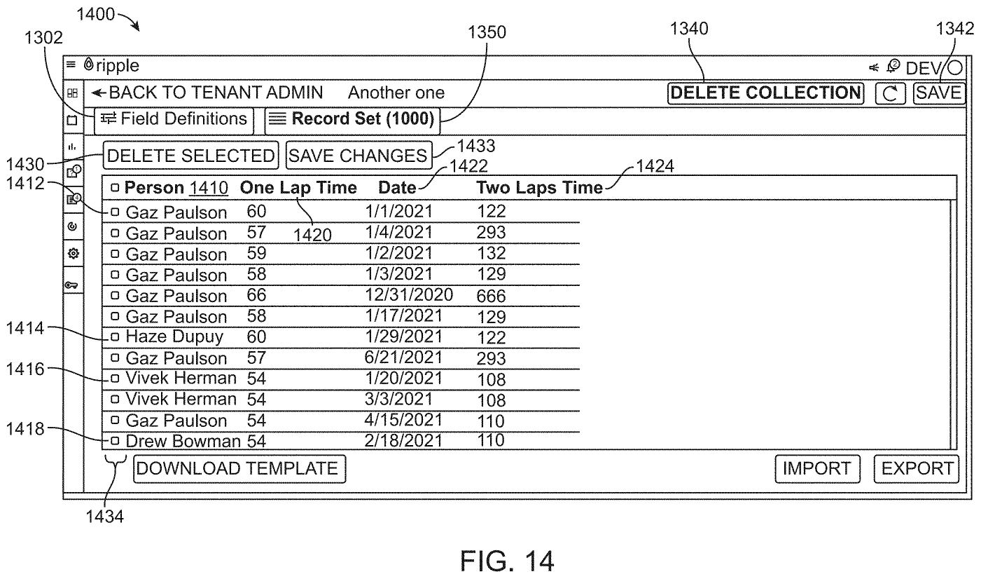

In some embodiments, the data sources from which the data is retrieved are user-configured. In some aspects, the computing system displays, via a GUI (e.g., a fourth GUI), an interactive element (e.g., drop-down, text-entry field, etc.) for configuring a source of the input data and receives, via the GUI, user input configuring the source of the input data. For example, a user can interact with a GUI to establish that a particular wearable device (e.g., based on a unique identifier of the wearable device) should be used to gather speed and heartrate data for a particular target entity. As another example, user input establishes that how the user is feeling should be gathered from a particular application that gathers survey data from the target entity on their mobile device. A user can further interact with the GUI to establish a format in which the data is stored (e.g., string, integer, numeric, etc.) and where the data is stored (e.g., in a remote database or on a user device, in certain fields, etc.). An example user interface for configuring data sources is shown in FIG. 10. The input data is retrieved and stored by the server computer based on the configured source. The input data can further be stored in a particular manner based on user configuration via interfaces such as those depicted in FIGS. 13 and 14.

At step 1204, the system computes a metric. The metric represents how well a target entity is performing in a role, and can indicate the target entity's success in the organization as a whole. The metric may be indicative of courses of action for improvement. The metric is computed based on the data retrieved at step 1202. In some embodiments, the metric is also based on the weights received via the GUI. For example, for the role of centerback on a soccer team, the configured skills and weights are defending (50%), passing (20%), and possession (30%). The system identifies defending data, passing data, and possession data. In some cases, the data is retrieved from a data store of the server computer, which periodically retrieves the data from remote devices at step 1202. Alternatively, or additionally, some data may be retrieved directly from a remote device in real-time (e.g., from a biometric sensor to assess the current physical status of the target entity).

The data is used to compute a metric, or representation of the entity's overall performance according to the selected skills and weights. In some examples, the metric is numeric, on some scale (e.g., 1-100, where 100 is best). As an example, the metric is given by:

0.5(DR+1/HRD)+0.2(PAR+1/HAR)+0.3(POT+1/POR),

where DR is a percentage of successful defense actions, HRD is an average measured heart rate associated with the defense actions, PAR is a percentage of successful passing actions, HAR is an average measured heart rate associated with the passing actions, POT is a time of possession, and POR is an average measured heart rate associated with the possession actions, each term being weighted using the respective configured weight.

In some implementations, computing the metric further includes analyzing the data based on how the data points for entities within a role or organization compare to one another. For example, the system computes the metric by analyzing different skills for a given role in turn for each of a set of entities assigned to that role. The server computer may identify a first skill value for a first skill for another entity in the role (e.g., a second entity different than the target entity). For example, the role is patrolling police officer and skills configured for the role include the skill categories of driving (including the skills of pit maneuvers, speed trials, etc.) and shooting (including the skills of target practice, gun safety, etc.). The server computer selects one of the skills (e.g., pit maneuvers) and identifies a score for the entity in pit maneuvers (e.g., an average score for a set of historical practice pit maneuvers for the entity). This may be repeated for each entity in the role (e.g., for all police officers in the role of patrolling officers an organization). In some implementations, the server computer generates a baseline value for the skill for the role. The baseline value may be set to the highest score for the skill, or a derivative thereof. For example, the server computer increments a first skill value equal to the highest score for the skill among entities in the role according to a predetermined margin to generate the baseline value. The predetermined margin may, for example, be 1% (e.g., the highest score for a basketball forward on a team is 83% shooting success, and a predetermined margin of 1% is added to establish a baseline value of 84% for the shooting skill). Upon determining the baseline value, the server computer compares the scores for other entities in the role to the baseline value. For example, the server computer identifies a skill value for the target entity, and other entities in the role (e.g., 75% shooting success, 39% shooting success, etc.). The server computer then computes a percentage of the baseline value for the skill for the other entities (e.g., for the target entity, the percentage of the baseline value for the shooting skill is (75/84)×100=89%). This may be repeated for each target entity in the role.

In some implementations, this process is repeated for each skill assigned to the role (e.g., for additional skills of speed trials, shooting, gun safety, etc., a respective baseline value is computed which is then compared to values for other entities in the role for that skill). For example, computing the metric further includes identifying a third skill value for a second skill for a third entity. The server computer moves on to another skill configured for the role, speed trials, for which a different entity in the role has a highest score of 95. This is incremented using the margin of 1% to arrive at a second baseline value of 96 for the speed trials skill. The server computer identifies a skill value for the speed trials skill (e.g., the second skill) for the target entity, and computes a percentage of the second baseline value for the skill value for the target entity. This can be repeated for each skill assigned to the role. In some implementations, if data is missing for a particular entity for a particular skill (e.g., if a striker has no data for tackle success rate), then the other skills are dynamically computed and reweighted to avoid counting this as a zero score. The server computer then computes the metric based on the percentage of the first baseline value and the percentage of the second baseline value. For example, the metric may be a weighted sum of the computed percentages, according to the user-configured weights for each of the skills. The scores can be recomputed and reweighted until every entity in the role is accounted for.

At step 1206, the server computer identifies an action likely to improve the metric. For example, the server computer may determine that the metric is below some threshold and perform further analysis to identify one or more skills in which the target entity is underperforming. As a specific example, if a player is underperforming in passing, scheduling passing practices may be identified as a corrective action, e.g., based on traversing a stored mapping of skills to actions. As another example, if an entity is showing signs of burnout, a more relaxed schedule may be identified as a corrective action.

At step 1208, the server computer triggers the action identified at step 1206. Triggering the action may include performing the action directly and/or causing another device or entity to perform the action. For example, the server computer identifies signs of distress in a police officer and determines that the officer's body cam should be turned on. The server computer triggers the action by transmitting a signal to the body cam, causing the body cam to activate.

Triggering the action in some examples includes modifying an entry on a calendar for the target entity to include an identified task to improve the performance. As an example, the identified action is to perform a particular training, and the server computer adds the training to the target entity's calendar. As another example, if it is found that the target entity has signs of burnout, then activities may be removed from the calendar, and/or therapy or meditation sessions are added to the calendar.

As another example, triggering the action includes transmitting an electronic mail (email) message including the metric and at least a subset of the input data or a derivative thereof. As a specific example, the system sends the metric, along with average passing and running scores, to a coach. The coach can then adjust an athlete's training regime based on the information in the email.

As another example, triggering the action includes displaying a GUI (e.g., a second GUI) including the metric and at least a subset of the input data or a derivative thereof. Example interfaces for presenting such results are illustrated in FIGS. 15-17. In some aspects, the computing system displays, via a GUI (e.g., a third GUI), performance metrics for a plurality of entities including the target entity and an attribute for each entity of the plurality of entities. The attribute can be some information about the target entity that is related to the metric, such as number of classes taken, hours worked per week, average running speed, and so forth. In some embodiments, the computing system displays an interface such as that shown in FIG. 17, showing attributes (e.g., minutes played as shown in FIG. 17) and metrics for a set of target entities. A user such as a coach can use such information to easily discern an appropriate intervention. For example, the server computer, by causing display of a user-friendly interface that shows the coach that a particular player with high scores and low playing time should play more, causes a modification of the attribute (in that the playing time is increased). Thus, the server computer, by displaying such an interface, causes a modification of the attribute for at least a subset of the plurality of entities.

At step 1210, the process returns to step 1202 and the metric is updated. The metric may, for example, be updated periodically. For example, new data is ingested daily, when new tests are taken, on a streaming basis, etc. The metric can be recomputed on a periodic basis so that the metric remains up-to-date. In some iterations, the action may not be triggered (e.g., if the target entity is performing well in all skills associated with their role).

Advantageously, the techniques of FIG. 12 distill data retrieved from one or more (often many) remote sources, which can be of many types. As shown in FIG. 3, such data in raw form is not very instructive as to how each entity is performing in a role or what to do to improve the performance of the entity and the overall organization. Using user-configured weights and skills, the system is able to identify performance metrics and recommended skills suitable for specific organizations and roles. Moreover, the results can be summarized in user-friendly GUIs that show a user information about the performance of the organization as a whole as well as providing the ability to drill down and understand the performance of individuals. This can give the user insights on how to make adjustment to improve performance. Additionally, the system can take automatic action without the need for user intervention. For example, the system can turn on or off a body camera, change schedules in a calendar, make a doctor's appointment, and other automatic interventions. These techniques provide multiple improvements to the functioning of a system for managing data for organizations, by efficiently distilling meaning from disparate and complex data and identifying and triggering appropriate interactions, which would otherwise involve complex computer aided and/or manual processes to attempt to identify performance issues from complex data coming from various sources.

FIGS. 13 and 14 illustrate example user interface views 1300 and 1400 illustrating data ingestion according to some embodiments. The user interface views of FIGS. 13 and 14 can be used to manage data ingestion (e.g., at step 1202 of the process 1200 of FIG. 12).

Referring to the user interface view 1300 shown in FIG. 13, data fields can be configured for the data retrieved and stored by the server computer. In the user interface view 1300, a Field Definitions tab 1302 has been selected. When the Field Definitions tab 1302 is selected, interface elements for configuring different fields are displayed, as shown in FIG. 13. In order to facilitate customization of the data fields, text boxes for Name 1304 and Data Type 1306 are presented in the user interface view 1300. In the example depicted in FIG. 13, the field names are Person 1310, One Lap Time 1312, Date 1314, and Two Laps Time 1316. For each of the named fields, a corresponding data type is configured—User 1320 for Person 1310, Number 1322 for One Lap Time 1312, Date 1324 for Date 1314, and Number 1326 for Two Laps Time 1316. This can be used to control the options for user configuration of values for each field as well as how the data is stored by the system. These data types can be selected using drop-down menus, as illustrated in FIG. 13. The user interface view 1300 further includes checkboxes which can be used to configure user-selected Key Fields 1330, Value Fact Dates 1332, and Facets 1334. The user interface view 1300 further includes a Delete Collection button 1340 for deleting the displayed field definitions and a Save button 1342 for saving the displayed field definition configurations. A Record Set tab 1350, when selected via user interaction, transitions the user interface to the user interface view 1400 depicted in FIG. 14.

Referring now to FIG. 14, corresponding data for each of the configured fields is shown for four athletes on a team (i.e., four entities in a role). The user interface view 1400 shows data for a set of entities, organized by the columns Person 1410, One Lap Time 1420, Date 1422, and Two Laps Time 1424. Under each column is a corresponding data element. For example, various One Lap Time 1420 values and Two Laps Time 1424 values are shown for each entity, with a date on which this data was collected. This data has been ingested from another computing device over a network and stored as structured data according to the configured fields. For a first entity, Gaz Paulson 1412, One Lap Time 1420 and Two Laps Time 1424 values are shown for several different dates 1422. For a second entity, Haze Dupuy 1414, One Lap Time 1420 and Two Laps Time 1424 values are shown for a date 1422. For a third entity, Vivek Herman 1416, One Lap Time 1420 and Two Laps Time 1424 values are shown for two dates 1422. For a fourth entity, Drew Bowman 1418, One Lap Time 1420 and Two Laps Time 1424 values are shown for a date 1422. This data shown in FIG. 14 can be ingested according to the configuration parameters established using the interface of FIG. 13. This structured data can then be used for computing the metrics for each of the entities as described herein. Using the user interface view 1400, a user can interact with checkboxes 1434 to select an entry. Interaction with a Delete Selected button 1430 will cause that entry to be deleted, and interaction with a Save Changes button will cause the changes to be saved. Thus, the user interface view 1400 can be used to view or delete entries. For example, if a particular entry appears to be erroneous (e.g., the one lap time is zero), or otherwise should be removed, the user can use the user interface view 1400 to remove one or more entries.

FIG. 15 illustrates an example user interface 1500 illustrating output based on a computed metric according to some embodiments. The output may be displayed, for example, to an administrator (e.g., a coach) via the second user device 108 of FIG. 1. The user interface 1500 shows information about a set of Users 1510 that are entities in a role, in particular, players on a sports team. The user interface 1500 displays a Coaches Dashboard 1502, which includes a Score Leader Board section 1550 and a Score Breakdown section 1555.

The Score Leader Board section 1550 shows a list of players on the team under a User 1510 column, according to a role under a Role Name 1512 column, along with a Latest Score 1514 (e.g. a metric, which can be computed as described above with respect to step 1204 of FIG. 12). The Users 1510 are further assigned a Latest Ranking 1516 based on the metric. For example, as shown in FIG. 15, a Latest Ranking 1516 of 1 is assigned to Users 1510 with the highest Latest Scores 1514, and a Latest Ranking 1516 of 5 is assigned to Users 1510 with the lowest Latest Scores 1514. A scale 1518 for the Latest Scores 1514 (e.g., with color coding) is also shown.

The Score Breakdown section 1555 shows factors contributing to the respective scores. Entities are shown in the User 1510 column, sorted by respective Role Names 1512. For each entity named in the User 1510 column, scores 1524 are shown, with bar chart and numeric formats to clearly show to the user how the scores compare for each entity and skill. Category Names 1520 are shown in one column, with skills in each category shown in a Field 1522 column. The appropriate Fields 1522 and Category Names 1520 vary depending on the Role Name 1512. For example, example, the midfielders have metrics (i.e., the Latest Scores 1514 shown on the left) based on individual scores for tackle success rate, distance travelled, passing success rate, possession success rate, and shot success rate. A coach can use this information to identify key areas that need improvement and take an appropriate action such as scheduling additional training for a target entity, assign a target entity to a different role on the team, and so forth. The user interface 1500 includes drop-down menus 1530, 1532, and 1534 that a user can interact with to filter by User 1530, Category Name 1532, or Role Name 1534. A scale 1536 for the scores 1524 (e.g., with color coding) is also shown.

FIG. 16A, FIG. 16B, and FIG. 16C illustrate additional example user interfaces 1602, 1604, and 1606, respectively. The user interfaces 1602, 1604, and 1606 illustrate output based on a computed metric according to some embodiments.

Referring to FIG. 16A, the first user interface 1602 is displayed on a computing device 1610 (e.g., a desktop or laptop computer). The user interface 1602 shows a recommended intervention 1612. Based on the ingested data, derivatives thereof, and/or computed metric, the system has determined that the target entity should improve their ability to handle stress 1614. This recommended intervention 1612 is displayed, along with a training plan 1616 (understand the effects of stress and learn how to manage it), and e-courses 1618a, 1618b (an active coping and problem solving e-course 1618a and a shifting unhelpful behaviors e-course 1618b). The first user interface 1602 shows that the training plan has been completed, as indicated by the check box 1620. The e-courses are associated with selectable interface elements for assigning the e-courses (assign buttons 1620a, 1620b). This first user interface 1602 can be displayed to an administrator (e.g., via second user device 108) to manage tasks assigned to an entity in a role that has been identified as one that would benefit from improvement in a particular skill or set of skills.

Referring to FIG. 16B, the second user interface 1604 shows a training plan 1630. A training plan 1630 may, for example, be displayed to a target entity that has been identified for an action to improve performance in one or more skills for a role. The second user interface 1604 shows the training plan 1630 “understand the effects of stress and learn how to manage it,” a progress bar 1632 indicating 100% progress, and trainings 1634 in the training plan 1630. The trainings 1634 can be navigated through by swiping the screen. One of the trainings 1634 is shown, a video 1636 “communicating effectively in the workplace,” which can be played by interacting with the video embedded in the user interface 1604. In some implementations, upon determining that the target entity should improve stress management, the system takes the action of presenting the user interface 1604 to the target entity, thereby causing the target entity to complete the training plan and improve their stress management skill level.

Referring to FIG. 16C, the third user interface 1606 shows a dashboard 1650. The dashboard 1650 shows a scorecard 1652 for a target entity, Joseph M. 1654, in the role of defender 1656. The dashboard 1650 shows a scorecard 1652 indicating a metric 1660 computed indicating the target entity's performance in the role of defender—55 is the metric 1660 computed for the target entity, and a goal metric value 1662 of 63 (e.g., a baseline metric corresponding to a highest performer in the role) and average metric value 1664 of 54. The dashboard 1650 also shows different skill categories for Joseph M. 1654 and corresponding scores—80% for technical 1670, 100% for tactical 1672, 60% for physical 1674, and 45% for physiological 1676. The scores are also displayed in association with symbols—a thumbs up 1680 indicating that the performance is on target (for technical and tactical which are above some threshold) or a caution sign 1682 indicating that the performance can use improvement (for physical and physiological which are scored below some threshold). The user interface 1606 further includes announcements 1690 (e.g., a practice update) and a to-do list 1692 (e.g., daily wellness survey and dribbling drill). The to-do list 1692 can include interventions that the system has identified as likely to improve the target entity's performance/performance metric. The dashboard 1650 can be presented to a target entity, causing the target entity to perform activities and improve their performance.

FIG. 17 illustrates another example user interface 1700 illustrating a coaches dashboard 1702 showing output based on a computed metric according to some embodiments. The output may be displayed, for example, to an administrator (e.g., a coach) via the second user device 108 of FIG. 1. The user interface 1700 shows information about a set of entities in a role, in particular, players on a sports team. The user interface 1700 shows an impact vs. playing time 1704 for each player 1706. A role 1708 is shown for each player 1706.

The user interface 1700 shows minutes played 1710 for each player 1706, indicating the correlation between the minutes played 1710 and the computed metric for each player 1706, as indicated by the ranked scores 1712 and raw scores 1714 for each player 1706. This allows the administrator to drill down to see how each player is being utilized and reshuffle as appropriate. For example, a coach may see that one of the best players is spending a lot of time on the bench and take action to increase the playing time for that player. Alternatively, or additionally, the system can perform such actions automatically, e.g., by updating an electronic calendar or modifying a starting lineup in an electronic document. On hover 1720, the user interface 1700 transitions to show additional information about a selected data element—here, the hover 1720 is on the minutes played for Theun Leclerc, and a pop-up shows information for that player.

It should be appreciated that the computing system for performance management may have one or more microprocessors/processing devices that can further be a component of the overall apparatuses. The control systems are generally proximate to their respective devices, in electronic communication (wired or wireless) and can also include a display interface and/or operational controls configured to be handled by a user to monitor the respective systems, to change configurations of the respective systems, and to operate, directly guide, or set programmed instructions for the respective systems, and sub-portions thereof. Such processing devices can be communicatively coupled to a non-volatile memory device via a bus. The non-volatile memory device may include any type of memory device that retains stored information when powered off. Non-limiting examples of the memory device include electrically erasable programmable read-only memory (“ROM”), flash memory, or any other type of non-volatile memory. In some aspects, at least some of the memory device can include a non-transitory medium or memory device from which the processing device can read instructions. A non-transitory computer-readable medium can include electronic, optical, magnetic, or other storage devices capable of providing the processing device with computer-readable instructions or other program code. Non-limiting examples of a non-transitory computer-readable medium include (but are not limited to) magnetic disk(s), memory chip(s), ROM, random-access memory (“RAM”), an ASIC, a configured processor, optical storage, and/or any other medium from which a computer processor can read instructions. The instructions may include processor-specific instructions generated by a compiler and/or an interpreter from code written in any suitable computer-programming language, including, for example, C, C++, C #, Java, Python, Perl, JavaScript, etc.

While the above description describes various embodiments of the invention and the best mode contemplated, regardless of how detailed the above text, the invention can be practiced in many ways. Details of the system may vary considerably in its specific implementation, while still being encompassed by the present disclosure. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

The teachings of the invention provided herein can be applied to other systems, not necessarily the system described above. The elements and acts of the various examples described above can be combined to provide further implementations of the invention. Some alternative implementations of the invention may include not only additional elements to those implementations noted above, but also may include fewer elements. Further any specific numbers noted herein are only examples; alternative implementations may employ differing values or ranges, and can accommodate various increments and gradients of values within and at the boundaries of such ranges.

References throughout the foregoing description to features, advantages, or similar language do not imply that all of the features and advantages that may be realized with the present technology should be or are in any single embodiment of the invention. Rather, language referring to the features and advantages is understood to mean that a specific feature, advantage, or characteristic described in connection with an embodiment is included in at least one embodiment of the present technology. Thus, discussion of the features and advantages, and similar language, throughout this specification may, but do not necessarily, refer to the same embodiment. Furthermore, the described features, advantages, and characteristics of the present technology may be combined in any suitable manner in one or more embodiments. One skilled in the relevant art will recognize that the present technology can be practiced without one or more of the specific features or advantages of a particular embodiment. In other instances, additional features and advantages may be recognized in certain embodiments that may not be present in all embodiments of the present technology.

What is claimed is:

1. A computer-implemented method comprising:
receiving, by a server computer from a remote device, input data associated with a performance of a target entity in a role in an organization;
receiving, by the server computer via input to a Graphical User Interface (GUI), a set of weights for a respective set of skills for the role;
computing, by the server computer, a metric based on the input data and the weights, the metric representing the performance in the role for the target entity;
identifying, by the server computer, an action likely to improve the metric; and
triggering, by the server computer, the action.

2. The method of claim 1, wherein triggering the action comprises one or more of:
modifying an entry on a calendar for the target entity to include an identified task to improve the metric;
transmitting an electronic mail (email) message including the metric and at least a subset of the input data or a derivative thereof;
displaying a second GUI including the metric and at least a subset of the input data or a derivative thereof; or
transmitting a suggestion thereby causing the target entity to perform the action.

3. The method of claim 1, wherein the input data includes one or more of:
biometric data received from a wearable device that collected the biometric data from the target entity;
performance data received from a computing device that analyzed performance of the target entity; or
survey or test data received from a user device that received responses from the target entity.

4. The method of claim 3, wherein the biometric data comprises one or more of heartrate data or blood oxygenation data.

5. The method of claim 1, further comprising:
displaying the GUI, the GUI comprising one or more interactive elements for modifying the weights;
receiving, via the one or more interactive elements, user input modifying the weights; and
updating the weights based upon the user input.

6. The method of claim 1, wherein computing the metric comprises:
identifying, by the server computer, a first skill value for a first skill for a second entity;
incrementing, by the server computer, the first skill value according to a predetermined margin to generate a first baseline value;
identifying, by the server computer, a second skill value for the first skill for the target entity; and
computing, by the server computer, a percentage of the first baseline value for the second skill value.

7. The method of claim 6, wherein computing the metric further comprises:
identifying, by the server computer, a third skill value for a second skill for a third entity;
incrementing, by the server computer, the third skill value according to the predetermined margin to generate a second baseline value;
identifying, by the server computer, a fourth skill value for the second skill for the target entity;
computing, by the server computer, a percentage of the second baseline value for the fourth skill value; and
computing, by the server computer, the metric based on the percentage of the first baseline value and the percentage of the second baseline value.

8. The method of claim 1, further comprising:
displaying, via a third GUI, performance metrics for a plurality of entities including the target entity and an attribute for each entity of the plurality of entities,
thereby causing a modification of the attribute for at least a subset of the plurality of entities.

9. The method of claim 1, further comprising:
displaying, via a fourth GUI, an interactive element for configuring a source of the input data; and
receiving, via the fourth GUI, user input configuring the source of the input data,
wherein the input data is retrieved and stored by the server computer based on the configured source.

10. The method of claim 1, wherein at least a subset of the input data is retrieved from a Global Positioning System (GPS).

11. A computing system comprising:
a processor; and
a non-transitory computer readable medium operatively coupled to the processor, the non-transitory computer readable medium comprising code executable by the processor for performing a method comprising:
receiving, by a server computer from a remote device, input data associated with a performance of a target entity in a role in an organization;
receiving, by the server computer via input to a Graphical User Interface (GUI), a set of weights for a respective set of skills for the role;
computing, by the server computer, a metric based on the input data and the weights, the metric representing the performance in the role for the target entity;
identifying, by the server computer, an action likely to improve the metric; and
triggering, by the server computer, the action.

12. The computing system of claim 11, wherein triggering the action comprises one or more of:
modifying an entry on a calendar for the target entity to include an identified task to improve the metric;
transmitting an electronic mail (email) message including the metric and at least a subset of the input data or a derivative thereof;
displaying a second GUI including the metric and at least a subset of the input data or a derivative thereof; or
transmitting a suggestion thereby causing the target entity to perform the action.

13. The computing system of claim 11, wherein the input data includes one or more of:
biometric data received from a wearable device that collected the biometric data from the target entity;
performance data received from a computing device that analyzed performance of the target entity; or
survey or test data received from a user device that received responses from the target entity.

14. The computing system of claim 13, wherein the biometric data comprises one or more of heartrate data or blood oxygenation data.

15. The computing system of claim 11, the method further comprising:
displaying the GUI, the GUI comprising one or more interactive elements for modifying the weights;
receiving, via the one or more interactive elements, user input modifying the weights; and
updating the weights based upon the user input.

16. The computing system of claim 11, wherein computing the metric comprises:
identifying, by the server computer, a first skill value for a first skill for a second entity;
incrementing, by the server computer, the first skill value according to a predetermined margin to generate a first baseline value;
identifying, by the server computer, a second skill value for the first skill for the target entity; and
computing, by the server computer, a percentage of the first baseline value for the second skill value.

17. The computing system of claim 16, wherein computing the metric further comprises:
identifying, by the server computer, a third skill value for a second skill for a third entity;
incrementing, by the server computer, the third skill value according to the predetermined margin to generate a second baseline value;
identifying, by the server computer, a fourth skill value for the second skill for the target entity;
computing, by the server computer, a percentage of the second baseline value for the fourth skill value; and
computing, by the server computer, the metric based on the percentage of the first baseline value and the percentage of the second baseline value.

18. The computing system of claim 11, the method further comprising:
displaying, via a third GUI, performance metrics for a plurality of entities including the target entity and an attribute for each entity of the plurality of entities,
thereby causing a modification of the attribute for at least a subset of the plurality of entities.

19. The computing system of claim 11, the method further comprising:
displaying, via a fourth GUI, an interactive element for configuring a source of the input data; and
receiving, via the fourth GUI, user input configuring the source of the input data,
wherein the input data is retrieved and stored by the server computer based on the configured source.

20. The computing system of claim 11, further comprising a Global Positioning System (GPS) from which at least a subset of the input data is retrieved.
